(19) United States
(12) Patent Application Publication
(10) Pub. No.: US 2011/0164127 A1
(43) Pub. Date: Jul. 7, 2011

(54) METHOD AND ENDOSCOPE FOR IMPROVING ENDOSCOPE IMAGES

(75) Inventors: Thomas Stehle, Hamburg (DE); Til Aach, Aachen (DE)

(73) Assignee: OLYMPUS WINTER & IBE GMBH, Hamburg (DE)

(21) Appl. No.: 12/997,432

(22) PCT Filed: Jun. 10, 2009
(86) PCT No.: PCT/EP09/04171

§ 371 Date: Mar. 9, 2011

(30) Foreign Application Priority Data
Jun. 12, 2008 (DE) 10 2008 027 905.6

(51) Int. Cl. H04N 7/18

(52) U.S. Cl. 348/65; 348/E07.085

(57) ABSTRACT

A method for improving endoscope images of, in part, fluorescent tissue regions which are illuminated with background light, which images are recorded by a color video camera wherein the color pixels from the endoscope images are transformed from the color space of the video camera (3) into a color space in which a straight line which passes through the color space region of the fluorescent light and the color space region of the background light runs parallel to a coordinate axis describing the fluorescent component of the pixels, and in that, in this color space, the fluorescent component of the pixels is changed by a non-linear characteristic which at least in regions increases the fluorescent contrast, namely the difference between higher fluorescent values and lower fluorescent values, and in that, finally, the pixels are transformed into a color space suitable for imaging.

### CROSS-REFERENCE TO RELATED APPLICATION

The present application is based upon and claims the benefit of priority from the PCT/EP2009/004171 filed on Jun. 10, 2009, the entire contents of which are incorporated herein by reference.

### BACKGROUND

1. Field

The present invention relates to endoscopic imaging, and particularly to methods and endoscopes for improving endoscopic images.

2. Description of the Related Art

Endoscopic images of fluorescent tissue regions in the human body are used to discover correspondingly marked tumours, e.g. in the bladder wall. Illuminating the tissue being examined using a background light, the wave length of which is distinctly removed from that of the fluorescent light, i.e. is easily distinguishable from it and which is also of lower brightness, in order not to swamp the very weak fluorescent light, in known from DE 199 02 184 C 1. Even so, it is always difficult to detect very weak fluorescence. Up to now, methods of improving such endoscope images have met with little success.

Methods for improving endoscope images by color space transformation are known from U.S. Pat. No. 4,805,016 A.

The task of the present invention consists in improving the detectability of fluorescence in the case of fluorescent images obtained by endoscope mentioned at the outset.

### SUMMARY

The endoscope images obtained by means of a video camera are usually within the RGB color space. According to the task of the invention, the fluorescence of these images is to be influenced but, if possible, the other image characteristics are to remain unchanged. First of all, the color pixels will be transformed in a color space, in which a straight line characteristic of the fluorescence, which, on the one hand, runs through the color space area of the background light and, on the other, through the color space area of the fluorescent light, is aligned parallel to a co-ordinate axis of this color space. Then, in this color space, the fluorescence can be influenced by adjustment along this co-ordinate axis, describing the fluorescence, without altering other image characteristics. It is now possible to convert the fluorescent component of the pixels by increasing the contrast of the fluorescent values using a non-linear characteristic. The pixels are then retransformed into a color space suitable to show the image, e.g. the normal RGB color space.

According to claim 2, the characteristic is beneficially developed in such a way that it raises the fluorescent values in the middle area, in a top section and lowers it in a lower section, where the fluorescence remains unchanged in the end areas of the characteristic. We therefore obtain a characteristic, which in the upper and lower end areas, lies on the identity straight lines and in between is essentially developed as an S-shape.

According to claim 3, the fluorescent contrast can be increased as time progresses. It is therefore possible to compensate for the gradual fading of the fluorescent substance, which in time leads to ever weaker fluorescent contrast.

A medical endoscope according to the invention is quoted in claim 4. According to the invention, this endoscope operates according to one of the methods indicated in claims 1 to 3, with respect to the image processing device.

### BRIEF DESCRIPTION OF THE DRAWINGS

The invention is shown diagrammatically and by way of example in the drawings.

FIG. 1 shows a diagrammatic view of an endoscope with image processing device and image display device,

FIG. 2 shows an enlarged diagrammatic view of the image processing device,

FIG. 3 is a diagram of the fluorescent characteristic used,

FIG. 4 shows a diagram of the light components used and,

FIG. 5 shows a representation of the image pixels in the RGB color space.

### DETAILED DESCRIPTION

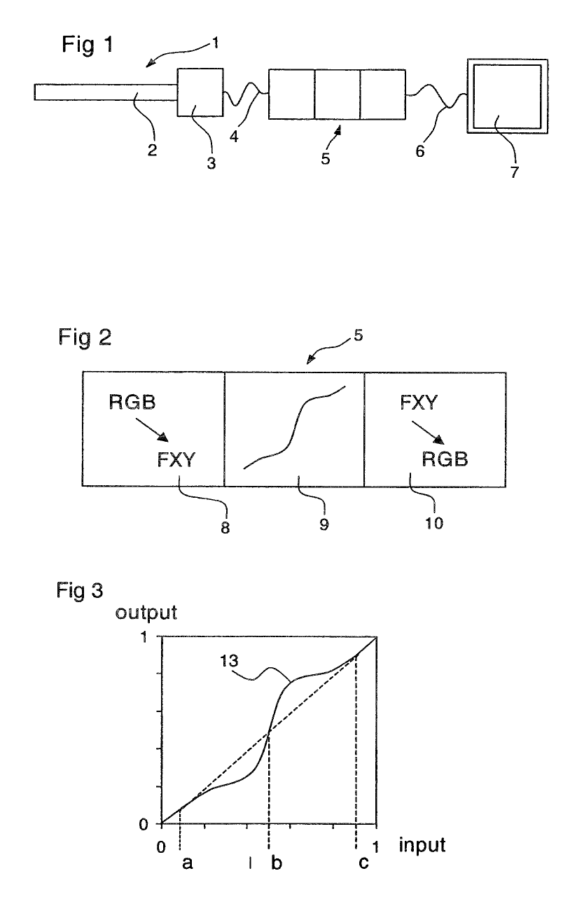

FIG. 1 shows a medical endoscope 1 with an elongated shaft 2, at the proximal end of which is located a color video camera 3. In another embodiment, the camera 3 may also be located in the distal end area of the shaft 2 directly behind the lens provided there. The color video camera 3 is connected to a line 4, which is used to transmit data and, for example, also to supply electricity, having an image processing device 5 in order to supply this image data. The image processing device 5 is connected by a line 6 to an image display device 7, e.g. a commercial monitor.

The endoscope 1 can be used, for example, in urology to examine the bladder wall for tumours and for this purpose is introduced by a shaft 2 through the urethra into the bladder (not shown). The image viewed by the video camera 3 is recorded, transmitted to the image processing device 5, where it is processed and is then displayed on the image display device 7.

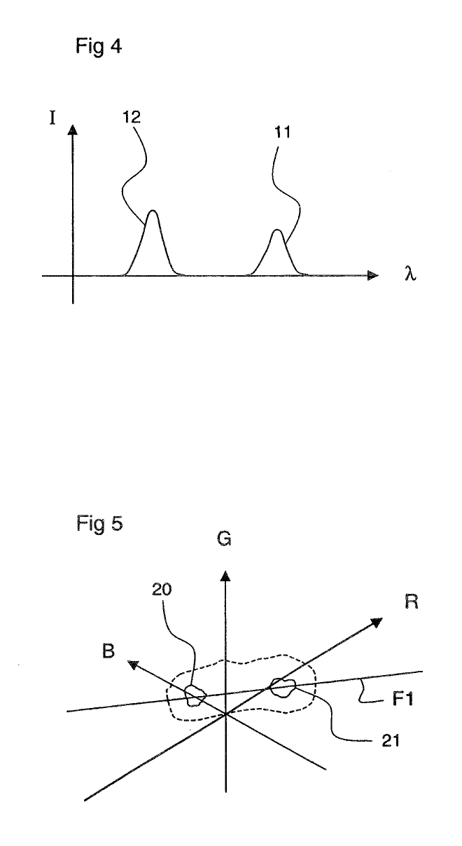

The endoscope 1 is used to examine tissue surfaces, e.g. the bladder wall, for any tumours, which are marked with a fluorescent substance. As shown in FIG. 4, where the light intensity I is plotted against the wavelength λ, the fluorescent tissue emits light in the area 11. The entire surface viewed is illuminated by background light in area 12, i.e. at a different wave length. The area 11 is usually in the red and the area 12 in the blue. Reference is made to DE 199 02 184 C 1 for details of this.

The pixels of an image recorded by the video camera 3 lie in the RGB color space in a cloud, as shown, for example, in outline by the dotted line in FIG. 5. This cloud has characteristic centroids in the color spaces 20 and 21, as shown in an example in FIG. 5. The color space 21 lies in the red and corresponds to the fluorescent light in the area 11 of FIG. 4. The color space area 20 lies in the blue in area 12 of FIG. 4.

In FIG. 5 a straight line is shown by F1, which runs through the color space areas 20 and 21 and normally at an angle in relation to the co-ordinate axes R, G, B. The straight line F1 runs through the color space area 20 with plenty of background light and little fluorescent light and through the area 21 with plenty of fluorescent light and little background light. In other words, different fluorescent values can be shown along this straight line F1, between the color space areas 20 and 21. Projecting a color vector onto the straight line F1 therefore provides a measure of the fluorescence.

FIG. 2 shows image processing 5 in detail. It has three stages 8, 9 and 10 in which the pixels of the image are processed one after the other.

In the first stage 8, pixels from the color space used by the camera, said color space being as a rule an RBG color space, are converted is succession to a different color space, which is described as FXY. The co-ordinate axes X and Y are unimportant. They merely have to be selected in such a way that a three-dimensional color space is fixed by F, X and Y. What is important is the position of the co-ordinate axis F, which must be placed parallel to the straight lines F1 of FIG. 5, and which therefore indicates the fluorescent component of a pixel in the new color space FXY. The color space FXY ensues from the original RGB color space, i.e. as a result of rotating and if necessary, shifting.

The second image processing stage 9 is used to change the fluorescence value non-linearly. In stage 10 a conversion is then made from the FXY color space to a color space usually used to display images, which in turn is usually the RGB color space.

FIG. 3 shows in greater detail, the characteristic line 13, which is used in image processing stage 9 to influence the fluorescent values. The aim of this conversion is to improve the visibility of the fluorescent light, which is very weak. The other image characteristics are to be altered as little as possible. This is achieved by converting the images only on the F co-ordinate, which is independent of the other co-ordinates. In other words, the fluorescence can be very heavily influenced, without otherwise altering the image impression.

When influencing the fluorescence, the characteristic line 13, shown as an example in FIG. 3, is used. At both ends of the characteristic line, i.e. in the areas 0 to a, where blue light is clearly visible, or in the area c to 1, where fluorescent light (red) is clearly visible, nothing is changed. In the area a to c in between, the fluorescence is reduced in the lower section a to b, in other words in the weakly blue area, and the blue portion is intensified, whilst in the upper section from b to c, the fluorescence (red) is increased.

This means that in areas with small fluorescent components, which are difficult to see, the fluorescent contrast is increased. By this arrangement, the image statement is improved above all in the areas of moderate fluorescence, where it is difficult to tell whether there is fluorescence present or not.

In the embodiment discussed, the background light 12 is in the blue and the fluorescent light 11 in the red. The color of the fluorescence may also be different, depending on the fluorescent dye. The background light may also be chosen differently, provided it only corresponds to an area region that is defined to some extent in the color space.

The fluorescent substance used to mark the tumour to be displayed, can lose its effect in time, so that the fluorescence diminishes. In order to compensate for this, the characteristic line 13 can be changed over time, so that the fluorescent contrast is increased as the substance fades or becomes less over time and the result is that, the fluorescent impression essentially remains the same.

1. A method for improving endoscope images of, in part, fluorescent tissue regions which are illuminated with background light, which images are recorded by a color video camera, wherein, the color pixels from the endoscope images are transformed from the colour space of the video camera into a color space in which a straight line which passes through the color space region of the fluorescent light and the color space region of the background light runs parallel to a coordinate axis describing the fluorescent component of the pixels, and in that, in the color space, the fluorescent component of the pixels is changed by a non-linear characteristic which at least in regions, increases the fluorescent contrast by increasing the difference between higher fluorescent values and lower fluorescent values and the pixels are transformed into a color space suitable for imaging.

2. The method according to claim 1, wherein the characteristic is developed such that no change occurs in the upper and lower end regions and in the region in between in an upper section of the characteristic an intensification occurs and in a lower section a weakening occurs.

3. The method according to claim 1, wherein the characteristic is changed over time such that fluorescent contrast increases over time.

4. A medical endoscope having a color video camera, an image processing device connected to the color video camera and an imaging device connected to the image processing device, wherein the image processing device is configured to carry out the method according to claim 1.
